(19) United States
(12) Patent Application Publication
(10) Pub. No.: US 2025/0180566 A1
(43) Pub. Date: Jun. 5, 2025

(54) Method of Quantifying Antigen-Binding Molecule Bound to Cells

(71) Applicants: SHIMADZU CORPORATION, Kyoto (JP); NATIONAL CANCER CENTER, Tokyo (JP)

(72) Inventors: Hirobumi FUCHIGAMI, Kashiwa-shi (JP); Masahiro YASUNAGA, Kashiwa-shi (JP); Shigehiro KOGANEMARU, Kashiwa-shi (JP)

(73) Assignees: SHIMADZU CORPORATION, Kyoto (JP); NATIONAL CANCER CENTER, Tokyo (JP)

(21) Appl. No.: 18/957,441

(22) Filed: Nov. 22, 2024

(30) Foreign Application Priority Data
Nov. 30, 2023 (JP) 2023-202878
Oct. 21, 2024 (JP) 2024-185123

(51) Int. Cl. G01N 33/574; G01N 33/68

(52) U.S. Cl. CPC G01N 33/57492; G01N 33/6848

(57) ABSTRACT

Provided is a method of quantifying an antigen-binding molecule bound to cells, comprising: a preparation step of preparing a biological sample derived from the cells, wherein the biological sample is homogenized, the cells express a target antigen bound specifically to the antigen-binding molecule, and the cells have been exposed to the antigen-binding molecule; an extraction step of adding an extraction solution to the biological sample to obtain an analyte, wherein the extraction solution comprises an organic acid and a nonionic surfactant, and the extraction solution has a pH of 1 or more and 3 or less; and a quantification step of analyzing the analyte to quantify the antigen-binding molecule.

### CROSS REFERENCE TO RELATED APPLICATIONS

This nonprovisional application is based on Japanese Patent Application No. 2023-202878 filed on Nov. 30, 2023 with the Japan Patent Office, the entire contents of which are hereby incorporated by reference.

### BACKGROUND OF THE INVENTION

### Field of the Invention

The present invention relates to a method of quantifying an antigen-binding molecule bound to cells.

### Description of the Related Art

In recent years, an antibody-based pharmaceuticals (hereinafter, sometimes referred to as “antibody drugs”) have been actively developed. Each antibody is characterized by binding specificity to a specific antigen (antigen specificity). Taking advantage of this characteristic, for example, an antibody that binds only to a receptor (antigen) specifically expressed on cancer cells may be administered to cancerous tissues containing such cancer cells to thereby induce immune responses to treat cancer. For example, cetuximab is known as an anticancer drug that specifically binds to the epidermal growth factor receptor (EGFR) and inhibits the growth of cancers such as colorectal cancer.

### SUMMARY OF THE INVENTION

In general, antibody drugs first each bind to their target antigen in order to exert their medicinal effects. Thus, it is desirable to develop a technology for precisely measuring the accumulation level of antibody that has reached the target tissue or target cells in order to evaluate the antibody drug under development. For example, the “YAKUGAKU ZASSHI” (Journal of Pharmaceutical Society of Japan), 2017; 137 (5): 535-544 discloses a technology for obtaining an image of molecules of antibody drug conjugate (ADC) distributed in a tissue, the technology including measuring the amount of the antibody drug conjugate (ADC) distributed in a tissue by using a mass spectrometer while using, as a reference, an imaging data obtained with a microscope and sharing the XY axis direction with the mass spectrometer. Clin Cancer Res. 2021 Jul. 15; 27 (14): 3970-3979 discloses a technology for analyzing, from a stained tissue image, the accumulation level of ADC in formalin-fixed, paraffin-embedded tissue sections (FFPE tissue sections) by using an anti-human antibody and an anti-payload antibody.

However, the conventional technologies indirectly detect the antibody bound to its target tissue, leaving room for improvement in terms of quantitative precision. The present invention has been made in light of the situations. An object of the present invention is to provide an accurate and sensitive method of quantifying an antigen-binding molecule bound to cells expressing its target antigen.

As a result of intensive research, the present inventors have found that an antigen-binding molecule bound to cells can be accurately quantified by adding a given extraction solution to a biological sample derived from the cells expressing its target antigen to prepare an analyte, and thus have completed the present invention.

The first aspect of the present invention provides a method of quantifying an antigen-binding molecule bound to cells, comprising:
a preparation step of preparing a biological sample derived from the cells, wherein
   the biological sample is homogenized,
   the cells express a target antigen bound specifically to the antigen-binding molecule, and
   the cells have been exposed to the antigen-binding molecule;
an extraction step of adding an extraction solution to the biological sample to obtain an analyte, wherein
   the extraction solution comprises an organic acid and a nonionic surfactant, and
   the extraction solution has a pH of 1 or more and 3 or less; and
a quantification step of analyzing the analyte to quantify the antigen-binding molecule.

The above and other objectives, features, aspects, and advantages of the present invention will become apparent from the following detailed description of the present invention as understood in connection with the accompanying drawings.

### BRIEF DESCRIPTION OF THE DRAWINGS

FIG. 1 is a schematic diagram illustrating a mode of the quantifying method according to an embodiment of the present invention.

FIG. 2 is graphs showing the results of LCMS analysis in Experiment 1.

FIG. 3 is graphs showing the results of LCMS analysis in Experiment 1.

FIG. 4 is a graph of the standard curve obtained from the results of analyzing the standard curve samples in Experiment 1.

FIG. 5 is graphs showing the results of LCMS analysis in Experiment 1.

FIG. 6 is graphs showing the results of quantifying an antibody bound to the surface of each cell line in Experiment 2.

FIG. 7 is a schematic diagram of the procedure for generating model tumors while using nude mice in Experiment 3 (left side) and images showing tissue staining of the collected model tumors (right side).

FIG. 8 is images showing the tissue staining of each model tumor collected in Experiment 3 (right side) and a graph showing the amount of antibody detected in each model tumor collected (left side).

FIG. 9 is graphs showing the amount of antibody detected in each collected model tumor (“TISSUE UNBOUND ANTIBODY”, “AMOUNT OF ANTIBODY IN WHOLE TISSUE”).

FIG. 10 is graphs showing the results of LCMS analysis in Experiment 4.

FIG. 11 is a graph of the standard curve obtained from the results of analyzing the standard curve samples in Experiment 4.

FIG. 12 is images showing the tissue staining of each model tumor collected in Experiment 5 (right side) and a graph showing the amount of antibody detected in each model tumor collected (left side).

FIG. 13 is graphs showing the amount of antibody detected in each collected model tumor (“TISSUE UNBOUND ANTIBODY”, “AMOUNT OF ANTIBODY IN WHOLE TISSUE”).

FIG. 14 is graphs showing the results of LCMS analysis in Experiment 6.

FIG. 15 is a schematic diagram of the procedure for generating model tumors while using nude mice in Experiment 7 (left side) and images showing tissue staining of the collected model tumors (right side).

FIG. 16 is images showing the tissue staining of each model tumor collected in Experiment 7 (right side) and a graph showing the amount of antibody detected in each model tumor collected (left side).

FIG. 17 is graphs showing the amount of antibody detected in each collected model tumor (“TISSUE UNBOUND ANTIBODY”, “AMOUNT OF ANTIBODY IN WHOLE TISSUE”).

### DESCRIPTION OF THE PREFERRED EMBODIMENTS

Hereinafter, an embodiment of the present invention (hereinafter, referred to as “this embodiment”) will be described. However, an embodiment of the present invention is not limited to this embodiment. As used herein, a notation in the form “A to Z” means the upper and lower limits of the range (i.e., A or more and Z or less). When no unit is stated for A with a unit stated only for Z, the unit for A is the same as that in Z.

### <<Method of Quantifying Antigen-Binding Molecule Bound to Cells>>

The first aspect of this embodiment provides a method of quantifying an antigen-binding molecule bound to cells, comprising:
a preparation step of preparing a biological sample derived from the cells, wherein
   the biological sample is homogenized,
   the cells express a target antigen bound specifically to the antigen-binding molecule, and
   the cells have been exposed to the antigen-binding molecule;
an extraction step of adding an extraction solution to the biological sample to obtain an analyte, wherein
   the extraction solution comprises an organic acid and a nonionic surfactant, and
   the extraction solution has a pH of 1 or more and 3 or less; and
a quantification step of analyzing the analyte to quantify the antigen-binding molecule.

### <Preparation Step>

In this step, a biological sample derived from cells is prepared. In this embodiment, the wording “biological sample derived from cells” means a biological sample that contains the cells themselves or materials (e.g., cell membrane, cell nucleus, cytoplasm) constituting the cells. The biological sample is homogenized. The term “homogenized” means that the components in the biological sample are homogeneous, and that the components are without non-homogeneous portion. The biological samples may be in solid state or in liquid state, but liquid state is preferred.

The cells express a target antigen bound specifically to the antigen-binding molecule. In this embodiment, the term “target antigen” means a protein mainly expressed on the surface of the above-mentioned cells. In one mode of this embodiment, the target antigen can also be understood as a “target biomolecule” or “target protein”. Examples of such a protein include a receptor expressed on the surface of the cells, an enzyme expressed inside the cells, a tumor necrosis factor receptor, insulin receptor, or a vascular endothelial growth factor receptor. Examples of the receptor expressed on the surface of the cells include epidermal growth factor receptor (EGFR), HER2, CD30, PD-L1, RANKL, a tumor necrosis factor receptor, insulin receptor, or a vascular endothelial growth factor receptor. In an aspect of this embodiment, the target antigen preferably includes at least one selected from the group consisting of EGFR, HER2, CD30, PD-L1, RANKL, tumor necrosis factor receptors, insulin receptor, and vascular endothelial growth factor receptors.

The cells have been exposed to the antigen-binding molecule. In this embodiment, the term “antigen-binding molecule” means a protein or peptide that binds specifically to the target antigen. Examples of the antigen-binding molecule include an antibody or a peptide aptamer. In one mode of this embodiment, it is preferred that the antigen-binding molecule should include an antibody or a peptide aptamer. In another aspect of this embodiment, the antigen-binding molecule preferably includes at least one selected from the group consisting of cetuximab, trastuzumab, brentuximab, denosumab, infliximab, adalimumab, etanercept, aflibercept, ramucirumab, atezolizumab, avelumab, and insulin analogs.

In this embodiment, the wording “have been exposed to the antigen-binding molecule” means that before the cells or the cells-containing tissue is collected, the cells have existed in an environment having the antigen-binding molecule or have been artificially exposed to the antigen-binding molecule. For example, the wording “have been exposed to the antigen-binding molecule” corresponds to culturing the cells in a medium containing the antigen-binding molecule or administering the antigen-binding molecule to the cells-containing tissue.

In this embodiment, the cells are preferably cells derived from cancer tissue, or cells derived from diseased tissue.

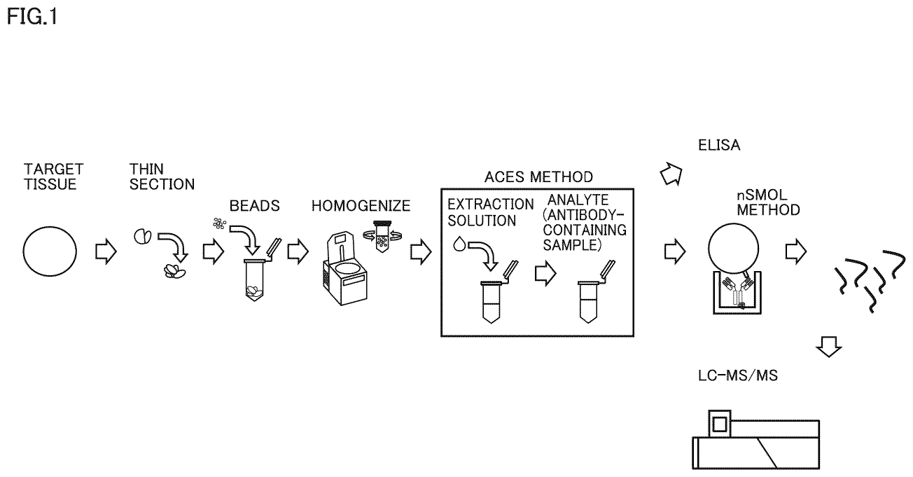

In this embodiment, the process of homogenizing the biological sample is not particularly limited and the sample may be homogenized by any known process. Examples of the homogenization process include: a process in which the cells or a tissue containing the cells (preferably, a tissue slice) is homogenized in a predetermined buffer (e.g., in D-PBS) by using beads for cell disruption (e.g., FIG. 1); a process by ultrasonic treatment; a process by freezing and thawing; and a process using a French press.

In one mode of this embodiment, the biological sample may further contain an enzyme inhibitor. The inclusion of the enzyme inhibitor in a biological sample makes it possible to inhibit degradation of the antigen-binding molecule by the enzyme. Examples of the enzyme inhibitor include a phosphatase inhibitor or a protease inhibitor.

### <Extraction Step>

In this step, an extraction solution is added to the biological sample to obtain an analyte. In this embodiment, the “extraction solution” means a solution used to dissociate the antigen-binding molecule bound to the target antigen from the target antigen. The extraction solution comprises an organic acid and a nonionic surfactant. In this embodiment, the “analyte” means a sample derived from the biological sample and used for analysis in the quantification step described below. Note that as used herein, the pretreatment method performed in the extraction step is sometimes referred to as the “ACES method” (Acidic Conditions Extraction supported with Surfactant method).

In this embodiment, the “organic acid” means an acidic organic compound. The organic acid is preferably an organic compound having a carboxy group. Examples of the organic acid include arginine, citrulline, or glycine. In one mode of this embodiment, the organic acid preferably comprises at least one selected from the group consisting of arginine, citrulline, and glycine. When the antigen-binding molecule is an antibody, it is preferable that the organic acid should be arginine in view of maintaining the stability of the antibody.

The concentration of the organic acid is preferably 20 mM or more and 1000 mM or less, and more preferably 100 mM or more and 500 mM or less based on the extraction solution.

In this embodiment, the “nonionic surfactant” means a surfactant that does not ionize when dissolved in water. Examples of the nonionic surfactant include an alkyl glycoside in which a sugar and a higher alcohol are linked via a glycosidic linkage. Examples of the alkyl glycoside include n-octyl-β-D-thioglucopyranoside, n-octyl-β-D-glucoside, n-octyl-β-D-maltoside, n-decyl-β-D-glucoside, n-decyl-β-D-maltoside, n-dodecyl-β-D-glucoside, n-heptyl-β-D-thioglucoside, and n-nonyl-β-D-thiomaltoside, trehalose C12. In one mode of this embodiment, the nonionic surfactant preferably includes an alkyl glycoside. The alkyl glycoside preferably includes at least one selected from the group consisting of n-octyl-β-D-thioglucopyranoside, n-octyl-β-D-glucoside, n-octyl-β-D-maltoside, n-decyl-β-D-glucoside, n-decyl-β-D-maltoside, n-dodecyl-β-D-glucoside, n-heptyl-β-D-thioglucoside, and n-nonyl-β-D-thiomaltoside. The alkyl glycoside more preferably includes n-octyl-β-D-thioglucopyranoside.

The concentration of the nonionic surfactant is preferably 0.1 mass % or more and 10 mass % or less, and more preferably 0.5 mass % or more and 5 mass % or less based on the extraction solution. In one mode of this embodiment, the concentration of the nonionic surfactant is preferably higher than the critical micelle concentration of the nonionic surfactant.

When the above nonionic surfactant may be n-octyl-β-D-thioglucopyranoside, the concentration of n-octyl-β-D-thioglucopyranoside is preferably 0.2 mass % or more and 10 mass % or less, and more preferably 0.5 mass % or more and 5 mass % or less based on the extraction solution.

The type and concentration of the organic acid and the type and concentration of the nonionic surfactant are determined by analyzing the extraction solution while using liquid chromatography and mass spectrometry.

The pH of the extraction solution is 1 or more and 3 or less, and preferably 1.5 or more and 2.5 or less. The pH of the extraction solution can be measured with a commercially available pH meter.

In this embodiment, the amount of the extraction solution added is not particularly limited as long as the effects of the present invention are exhibited. However, the amount, for example, is preferably 10 times or more and 50 times or less, and more preferably 20 times or more and 40 times or less based on the mass of the biological sample. In this case, the density of the extraction solution is assumed to be 1 mg/l μL, and the amount of the extraction solution added should then be set.

In this embodiment, the extraction solution may be added directly to the biological sample to obtain an analyte (e.g., FIG. 1). Alternatively, liquid and solid components were separated from the biological sample and recovered, and then the extraction solution may be added to the solid component to obtain an analyte. The method of separating the biological sample into liquid and solid components is not particularly limited, but may include separating the biological sample into supernatant (liquid component) and precipitate (solid component) by centrifugation.

In one mode of this embodiment, the extraction step may include: separating the biological sample into a liquid component and a solid component; adding the extraction solution to the solid component to obtain a first analyte; and obtaining the liquid component as a second analyte.

In other aspects of this embodiment, the preparation step and the extraction step may be performed simultaneously. For example, in a method, the extraction solution is added to the biological sample, followed by homogenization.

In one mode of this embodiment, the extraction solution may further contain an enzyme inhibitor, an internal standard, or an adsorption inhibitor. The term “adsorption inhibitor” herein refers to a reagent that inhibits the physical adsorption of the antigen-binding molecule on, for instance, the wall surface of a microtube. Examples of the enzyme inhibitor include a phosphatase inhibitor or a protease inhibitor. Examples of the internal standard include an isotope-labeled antigen-binding molecule. Examples of the adsorption inhibitor include BSA or mouse IgG.

### <Quantification Step>

In this step, the analyte is analyzed to quantify the antigen-binding molecule. In one mode of this embodiment, the wording “analyzing the analyte” includes directly analyzing the analyte or analyzing the analyte after removing a solid component (e.g., the residual precipitate) in the analyte.

In this embodiment, the method of quantifying the antigen-binding molecule may include directly detecting and quantifying the antigen-binding molecule, or detecting and quantifying a peptide(s) derived from the antigen-binding molecule.

In this embodiment, the method of quantifying the antigen-binding molecule is not particularly limited. Examples include an ELISA-based quantification method and liquid chromatogram tandem mass spectrometry (LC-MS analyzer or LC-MS/MS analyzer)-based quantification method.

The LC column in the LC-MS analyzer is not particularly limited, and hydrophobic columns (e.g., C30, C18, C8, C4) commonly used for protein or peptide analysis, or columns containing a hydrophilic affinity chromatography carrier can be selected, if appropriate, and used. The sample is optionally treated by, for example, desalting, solubilization, extraction, concentration, or drying, followed by mass spectrometry.

The ionization method in mass spectrometry is not particularly limited and examples of the method that can be adopted include electron ionization (EI), chemical ionization (CI), field desorption (FD), fast atom bombardment (FAB), matrix-assisted laser desorption/ionization (MALDI), electrospray ionization (ESI), and others. The method of analyzing an ionized sample is also not particularly limited, and the magnetic field deflection type, quadrupole (Q) type, ion trap (IT) type, time-of-flight (TOF) type, or Fourier transform ion cyclotron resonance (FT-ICR) type, for instance, can be selected, if appropriate, depending on the ionization method. MS/MS analysis, MS3 or higher multi-step mass spectrometry, or multiple reaction monitoring (MRM) can also be performed using, for instance, a triple quadrupole mass spectrometer.

The equipment particularly suitable for the quantification method in this embodiment is not particularly limited. Examples include LCMS-8030, LCMS-8040, LCMS-8050, LCMS-8060, LCMS-9030, and LCMS IT-TOF (all manufactured by Shimadzu Corporation).

In one mode of this embodiment, when the antigen-binding molecule is an antibody, the quantification step preferably includes using the nSMOL method to limit peptides to those derived from the variable region of the antibody and analyzing the resulting peptide(s) by LC-MS/MS analysis. The nSMOL method can be performed using, for example, nSMOL Antibody BA Kit (trade name; manufactured by Shimadzu Corporation).

The conventional technologies indirectly detect an antigen-binding molecule (e.g., an antibody) bound to cells expressing its target antigen, leaving room for improvement in terms of quantitative precision. In the present invention, the antigen-binding molecule bound to the cells is recovered from the cells by using an extraction solution containing an organic acid and a non-surfactant, so that direct detection of the antigen-binding molecule is possible, thereby improving the accuracy of quantification. The quantification method of the present invention makes it possible to not only analyze the accumulation state of antigen-binding molecule (e.g., an antibody drug) in its target tissue (e.g., cancer tissue), but also analyze whether or not the antigen-binding molecule is accumulated in unintended tissues (e.g., normal tissue). Therefore, the quantification method of the present invention is also useful for elucidating the mechanism of side effects caused in normal tissues.

### EXAMPLES

Hereinbelow, the present invention will be described in more detail by way of Examples. However, the present invention is not limited to them.

### Experiment 1: Quantifying Antibody Present in Cancer Tissue

The following procedure was used to quantify an antibody present in cancer tissue. Here, the cancer tissue corresponds to a tissue containing cells expressing a target antigen, and the antibody corresponds to an antigen-binding molecule.

### (Example: Extraction by ACES Method)

### (Preparation Step, Extraction Step)

First, the following cancer tissue and preparation solution were provided.

### Cancer Tissue:

Cancer (esophageal cancer) tissues collected from patients administered with cetuximab and cancer tissues from experimental model mice administered with cetuximab

### (1) Tissue Washing Solution:

D-PBS
Phosphatase inhibitor (1:50 (volume ratio), PhosSTOP)
Protease inhibitor mix (1:50 (volume ratio), complete ULTRA)

### (2) Inhibitor Solution:

200 mM Arginine-HCl solution (pH 2.1)
Phosphatase inhibitor (1:50 (volume ratio), PhosSTOP)
Protease inhibitor mix (1:50 (volume ratio), complete ULTRA)

### (3) Internal Standard IS Solution:

Inhibitor solution
SILuMAb cetuximab (final concentration 0.25 μg/ml)
BSA (final concentration 0.1%)
Mouse IgG1 (clone 2545) (final concentration 50 μg/ml)

### (4) Extraction Solution:

200 mM Arginine-HCl solution (pH 2.1)
n-Octyl-β-D-thioglucopyranoside (final concentration 2%)

First, the cancer tissue was sliced using a slicer to prepare thin sections (5 μm thick). Thin sections (10 mg) were transferred to a low-absorption microtube, and a cooled tissue washing solution (200 μl) was added thereto. Tissue disruption beads (zirconia beads; 2 mm diameter; 8 beads) were further added to the microtube.

The cancer tissue in the microtube was crushed (homogenized) using a bead homogenizer (MicroSmash MS-100R) under conditions at 5,000 rpm×1 min under cooling (<2° C.). The microtube was then allowed to stand in the device for 1 minute and cooled. This operation was performed a total of four times. In this manner, a sample containing crushed cancer tissue (homogenized biological sample) was prepared (preparation step). The biological sample is a sample derived from cancer cells.

The whole volume of the sample containing crushed cancer tissue (homogenized biological sample) was transferred to another low-adsorption microtube. Tissue washing solution (100 μl) was further added to the microtube, and the crushed cancer tissue was washed and recovered (twice). The recovered sample was sonicated for 5 minutes in an ultrasonic cleaner under ice-cooling (0° C.) and defoamed. The microtube containing the sample was subjected to centrifugation (at 16,000 g×5 minutes and 4° C.) to separate the sample into supernatant and precipitate. The entire supernatant was recovered in another low-absorption microtube and used as a “free antibody sample” (second analyte). The “free antibody sample” was stored at 4° C.

The precipitate was admixed with an inhibitor solution (160 μl), an internal standard IS solution (40 μl), and an extraction solution (200 μl) to give a precipitate suspension. The resulting suspension was allowed to stand for 1 hour under ice-colding. The suspension was then resuspended by pipetting and allowed to stand at 4° C. overnight (extraction step; ACES method). The tube containing the suspension was subjected to centrifugation (at 16,000 g×30 minutes and 4° C.) to separate the suspension into supernatant and precipitate. The supernatant was recovered, and centrifugally filtered (at 10,000 g×2 minutes and 4° C.) using Ultrafree MC (0.22 μm) to obtain filtrate. The filtrate obtained was recovered in another low-absorption microtube and used as a “bound antibody sample” (analyte or first analyte). The bound antibody sample was stored at 4° C.

### (Quantification Step)

The following preparation solutions were provided.

### (1) Standard Diluent:

Inhibitor solution
BSA (final concentration 0.1%)
Mouse IgG1 (clone2454) (final concentration 50 μg/ml)

### (2) Washing Solution:

D-PBS
n-Octyl-β-D-thioglucopyranoside (final concentration 0.1%)

### (3) Neutralizing Solution:

250 mM Na₂HPO₄ solution
n-Octyl-β-D-thioglucopyranoside (final concentration 1%)

### (4) Reaction Solution:

25 mM Tris-HCl solution (pH 8.0)

### (5) Initial Mobile Phase:

MilliQ water (0.1% formic acid)+acetonitrile (volume ratio 99:1)

Standard curve samples and QC samples with the following compositions were provided.

The standard curve samples: all samples were prepared using a standard diluent.
Blank sample: 0 ng/ml cetuximab
Standard curve 1:313 ng/ml cetuximab
Standard curve 2:625 ng/ml cetuximab
Standard curve 3:1.25 μg/ml cetuximab
Standard curve 4:2.50 μg/ml cetuximab
Standard curve 5:5.00 μg/ml cetuximab
Standard curve 6:10.0 μg/ml cetuximab
Standard curve 7:20.0 μg/ml cetuximab
Standard curve 8:40.0 μg/ml cetuximab
The QC samples: all samples were prepared using a standard diluent.
LLOQ: 313 ng/ml cetuximab
LQC: 938 ng/ml cetuximab
MQC: 3.75 μg/ml cetuximab
HQC: 12.5 μg/ml cetuximab
HLOQ: 40.0 μg/ml cetuximab

Here, 20 μL each of the corresponding standard curve sample or QC sample was added to 160 μL of matrix solution (a solution in which foreign components derived from the tissue for measurement were extracted in the same manner as for the sample for measurement while using an inhibitor solution and an extraction solution). An additional 20 μL of internal standard IS solution was added. These samples correspond to the “standard curve sample (with IS)” and “QC sample (with IS)” described below. In addition, in the above preparation, samples further containing 20 μL of matrix solution instead of 20 μL of internal standard IS solution were also prepared. These samples correspond to the “standard curve sample (without IS)” and “QC sample (without IS)” described below.

The cetuximab (antigen-binding molecule) in each analyte was quantitatively analyzed by generating a peptide(s) specific for cetuximab (peptide(s) derived from the variable region) by the nSMOL method and detecting the peptide(s) with a mass spectrometer. Specifically, the procedure is as follows.

(Generating Peptides by nSMOL Method)

Bound antibody samples, standard curve samples (with or without IS), and QC samples (with or without IS) were each dispensed in 200 μl portions into low-absorption microtubes. Next, the washing solution (600 μl) and the neutralizing solution (200 μl) were added to each microtube.

In addition, 200 μl of the free antibody sample was transferred to a low-absorption microtube, and another 20 μl of the internal standard IS solution was added. Next, the washing solution (760 μl) and the neutralizing solution (20 μl) were added to the microtube.

Immunoglobulin collection resin in nSMOL Antibody BA Kit (Shimadzu Corporation) was suspended well. Next, 12.5 μl thereof was collected and added to each tube. Each tube was stirred for 30 minutes in a self-standing tube mixer. Each tube was then subjected to centrifugation (at 15,000 g×5 minutes and 4° C.). From each tube, 700 μl of supernatant was gently removed while taking care not to aspirate any resin. The remaining suspension (resin and residual liquid) in each tube was transferred to Ultrafree MC (0.22 μm). To each tube after transferring the suspension was added a washing solution (300 μl), and the resin remaining in each tube was recovered and transferred to the Ultrafree MC (0.22 μm).

Centrifugal filtration was conducted (at 10,000 g×1 minute and 25° C.) on each Ultrafree MC to remove filtrate. Washing solution (300 μl) was added to each Ultrafree MC, and centrifugal filtration (at 10,000 g×1 minute and 25° C.) was performed to remove the filtrate (twice in total). D-PBS (300 μl) was added to each Ultrafree MC and centrifugal filtration (at 10,000 g for 1 minute and 25° C.) was performed to remove the filtrate (three times in total).

The reaction solution (85 μl) was added to each Ultrafree MC. FG beads Trypsin DART in nSMOL Antibody BA Kit was well suspended. Then, 5 μl thereof was collected and added to each Ultrafree MC. The nSMOL reaction was then carried out by incubation at 52° C. for 5 hours under saturated vapor pressure.

Stop solution (10 μl) in the nSMOL Antibody BA Kit was added to each Ultrafree MC to stop the nSMOL reaction. Centrifugal filtration (at 10,000 g×1 minute and 25° C.) was then performed on each Ultrafree MC, and the entire filtrate was recovered in another microtube. Each microtube was placed on a magnetic stand and allowed to stand for 2 minutes to remove excess magnetic beads. The supernatant (90 μl) in each microtube was then recovered into a low-absorption microtube. The initial mobile phase (90 μl) was further added to the low-absorption microtube. The resulting mixture was placed in a polypropylene vial for HPLC and air bubbles were removed. In this manner, peptides specific for cetuximab (peptides derived from the variable region) generated.

### (Detecting Peptide(s) by Mass Spectrometry)

The peptide specific for cetuximab (SEQ ID NO: 1: ASQSIGTNIHWYQQR (one letter code)) was quantified by LCMS analysis under the conditions shown below.

### List of Solutions Prepared:

Mobile phase A: MilliQ (LCMS grade)
Mobile phase B: 0.1% formic acid+acetonitrile (LCMS grade)

### LCMS Analysis Conditions:

Column used: Shimpack GISS C18, 2.1 mm ID×50 mm
Column temperature: 50° C.
Sample injection volume: 10 μL
Nebulizer gas flow rate: 3 l/min
Drying gas flow rate: 10 l/min
Heating gas flow rate: 10 l/min
ESI interface temperature: 300° C.
Heat block temperature: 400° C.
DL temperature: 250° C.
CID gas pressure: 270 kPa

### HPLC Conditions:

HPLC: Shimadzu Nexera X2
Flow rate: 0.4 ml/min
0-1.5 min: 1% B
1.5-4.9 min: 1-38% B Gradient
4.9-6.5 min: 38% B
6.5-7.0 min: 38-95% B Gradient
7.0-9.0 min: 95% B
9.0-9.5 min: 95-1% B
9.5-13.5 min: 1% B
LCMS analysis time: 3.0-6.0 min

### LCMS MRM Analysis Transition Conditions:

LCMS: Shimadzu LCMS-8060
Cetuximab, ASQSIGTNIHWYQQR
596.95 (+++)>651.80 (y10++) for quantification
596.95 (+++)>751.85 (y12++) confirmation ion
596.95 (+++)>708.35 (y11++) confirmation ion
13C6, 15N4-Cetuximab, 13C6, 15N4-ASQSIGTNIHWYQQR
600.45 (+++)>657.10 (y10++) for quantification (Ab_IS)
600.45 (+++)>757.30 (y12++) confirmation ion
600.45 (+++)>713.15 (y11++) confirmation ion

### Comparative Example

Thin sections (about 10 mg) of the cancer tissues were enzyme-treated using Protein Works Auto-eXpress Digest Kits (manufactured by Waters). The resulting enzyme-treated product was centrifuged (at 800 g×15 min and 10° C.) and the supernatant (about 160 μl) was recovered. The recovered supernatant was analyzed by LCMS analysis under the same conditions as above.

### (Results)

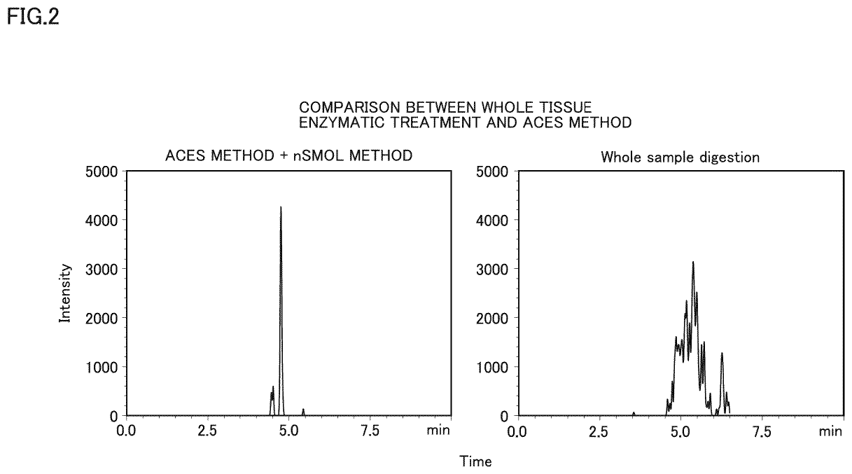

FIG. 2 shows the results of LCMS analysis of the bound antibody samples in the Example and the samples in the Comparative Example. In FIG. 2, the horizontal axis shows the retention time in LC, and the vertical axis shows the intensity of detection when the peptide corresponding to that retention time is analyzed by mass spectrometry. The results in FIG. 2 demonstrated that only the peptide specific to cetuximab was detected by the process using the ACES and sMOL methods (left graph in FIG. 2). In other words, cetuximab in cancer cells was able to be detected by the process in the Example. On the other hand, multiple peaks were detected in the sample obtained by enzymatic treatment of the whole cancer tissue (right graph in FIG. 2).

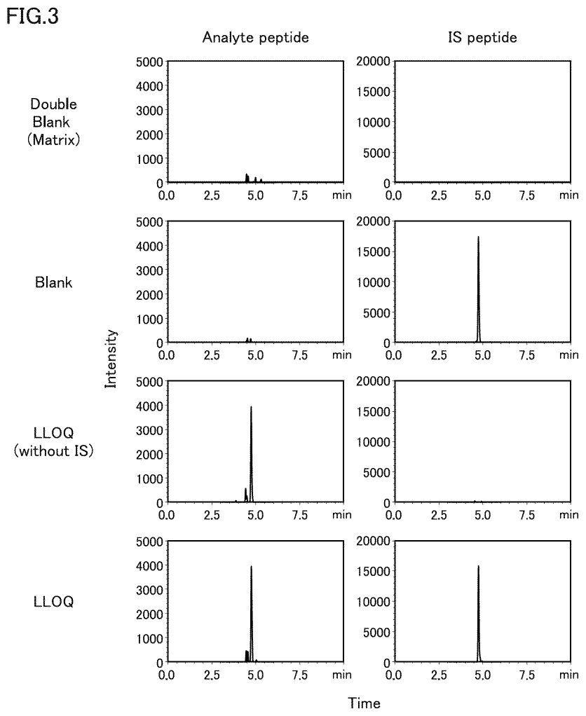

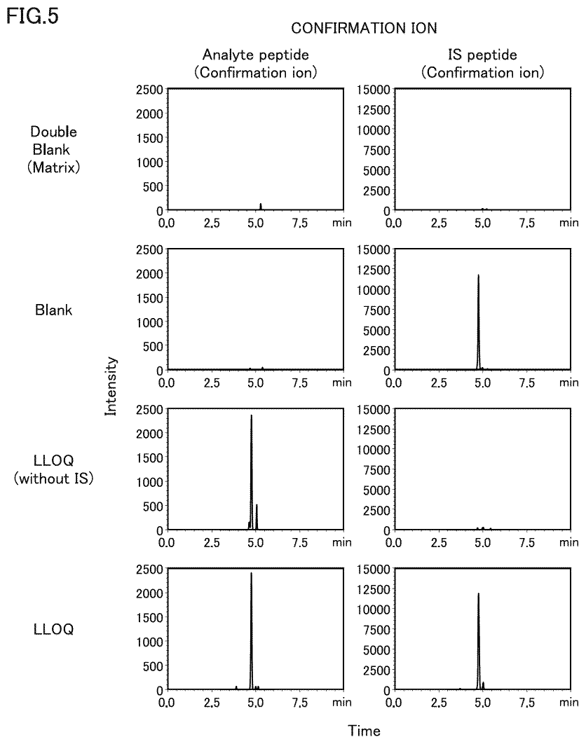

FIG. 3 shows the results of LCMS analysis of the standard curve sample (blank) (with IS (second graph from the top) or without IS (first graph from the top)) and the QC sample (LLOQ) (with IS (fourth graph from the top) or without IS (third graph from the top)) in the Example. The results of FIG. 3 demonstrated that cetuximab was detectable even at a concentration of 313 ng/ml under the analytical conditions of the Example. The results were likewise confirmed for the confirmation ion (596.95 (+++)>751.85 (y12++)) (FIG. 5).

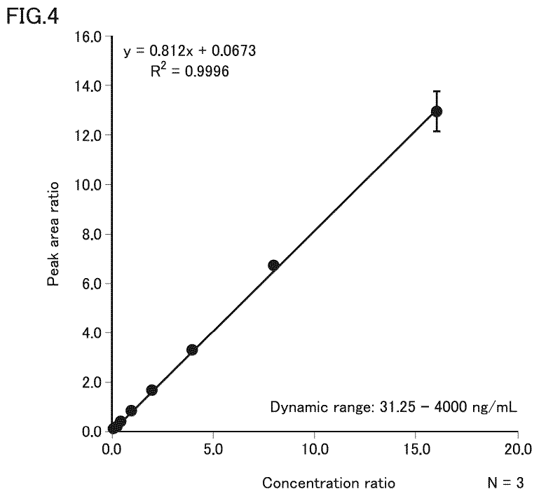

FIG. 4 shows a graph of the standard curve obtained from the results of analyzing the standard curve samples (blank, standard curves 1-8) (with IS) in the Example. In FIG. 4, the horizontal axis shows the concentration ratio of cetuximab in each standard curve sample (to the concentration of IS), and the vertical axis shows the ratio of the peak area of analyte to the peak area of IS in the mass spectrometry. The results in FIG. 4 suggested that it was possible to quantify cetuximab under the analytical conditions of the Example.

### Experiment 2: Quantifying Antibody Bound to Surface of Each Cell Line

Four cell lines (A-431, FaDu, TE-4, and SW620) were used to quantify an antibody bound to the surface of each cell line by the following procedure. First, respective cells were seeded at 4×10⁵ cells/well in a 12-well plate and allowed to adhere in an incubator (at 37° C. and 5% CO₂). One ml of 5 mg/ml (excess) cetuximab was added to each well and allowed to stand for 30 minutes under ice-cooling. After the reaction, cetuximab was removed by aspiration and each well was washed and aspirated twice using D-PBS.

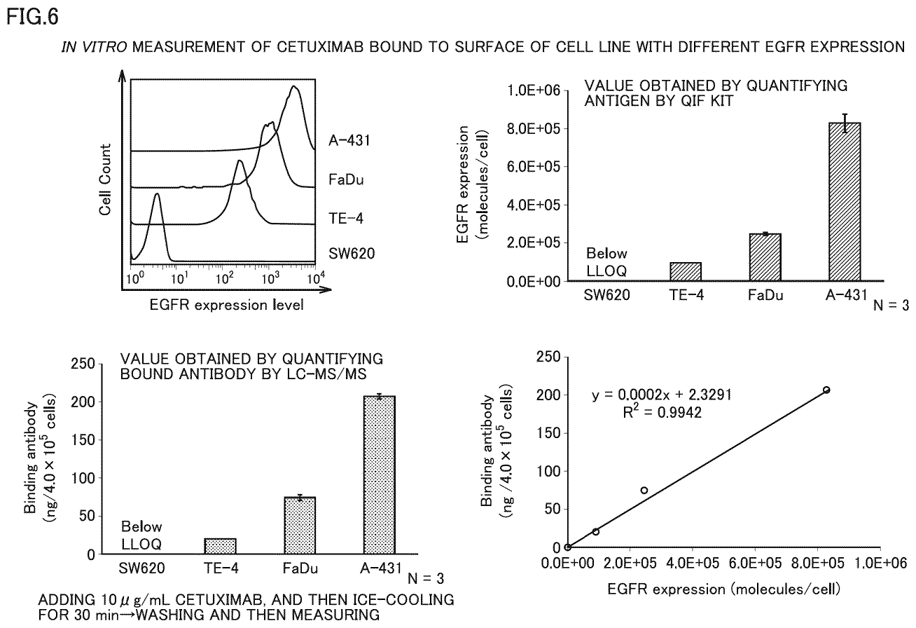

For each well, the inhibitor solution (160 μl), internal standard IS solution (40 μl), and extraction solution (200 μl) were added. The mixture was pipetted well, and allowed to stand for 1 hour under ice-cooling (extraction step, ACES method). The suspension was recovered into a tube, which was centrifuged (at 16,000 g×30 minutes and 4° C.) to separate into supernatant and precipitate. The supernatant was recovered and centrifugally filtered (at 10,000 g×2 minutes and 4° C.) using Ultrafree MC (0.22 μm) to obtain filtrate. Afterwards, the samples were processed and quantified in the same manner as for the quantification step in “Experiment 1: Quantifying antibody present in tissue sample”. The results are shown in FIG. 6 (lower left and lower right graphs). The results in FIG. 6 indicate that the antibody (cetuximab) bound to each cell surface can be quantified in correlation with the expression level of EGFR as quantified with a QIF kit (manufactured by DAKO) (upper left and right in FIG. 6).

### Experiment 3: Quantifying Antibody Bound to Tissue in Model Tumor

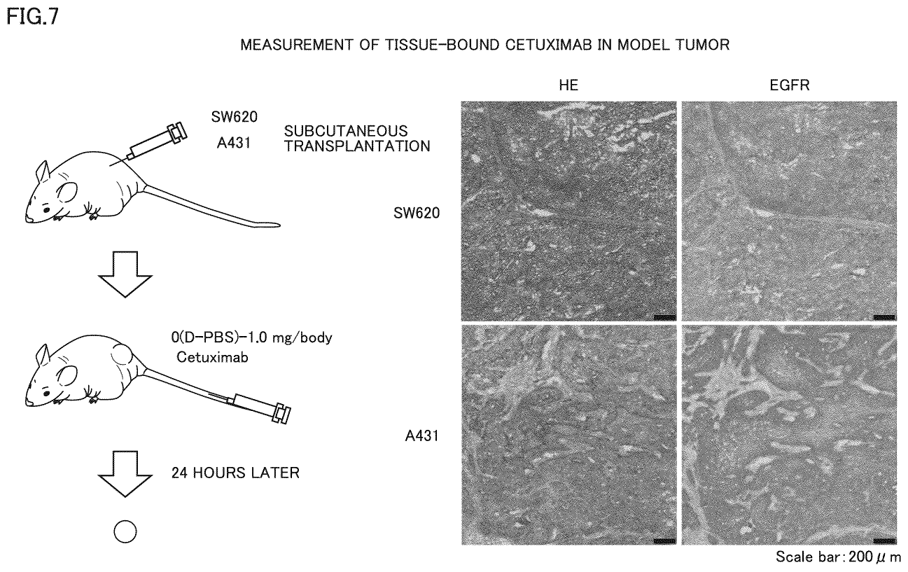

Nude mice (BALB/c, female, 5-8 weeks old) were subcutaneously transplanted with SW620 or A431 strain. At that time, the number of transplanted cells was 1×10⁷ cells per mouse. The mice were reared under normal conditions for 14 to 21 days until the transplanted cells engrafted. Then, cetuximab (0-1.0 mg/body) was injected intravenously into the mice. Twenty-four hours after intravenous injection, the transplanted cells (model tumors) were collected from the mice (FIG. 7, left side).

The collected model tumors were each tissue-stained with HE and anti-EGFR antibody (FIG. 7, right side). The model tumors collected from the mice transplanted with the A431 strain were found to express EGFR.

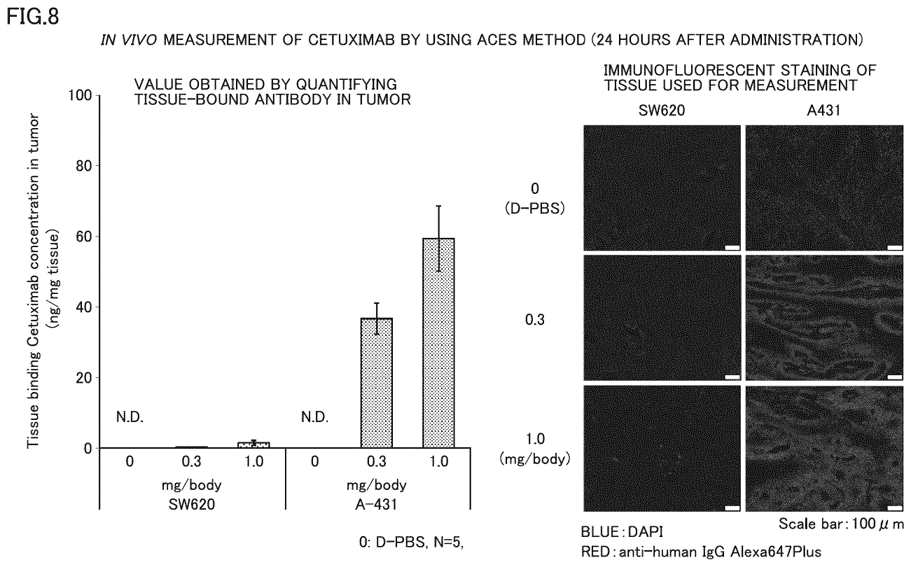

In addition, the collected model tumors were each tissue-stained with DAPI and anti-human IgG antibody (FIG. 8, right side). In model tumors collected from mice transplanted with the A431 strain, an increase in the amount stained with anti-human IgG antibody was observed depending on the dose of cetuximab administered.

The collected model tumors were treated by the same method as in Experiment 1, and the cetuximab bound to the model tumors was quantified. The samples used for quantification were those corresponding to the “bound antibody samples” in Experiment 1. FIG. 8 (left side) shows the results. In model tumors collected from mice transplanted with the A431 strain, an increase in the amount of cetuximab quantified using the ACES method was observed depending on the dose of cetuximab administered. This result correlated with that of the conventional tissue staining (FIG. 8). On the other hand, cetuximab was hardly detectable in model tumors collected from mice implanted with the SW620 strain.

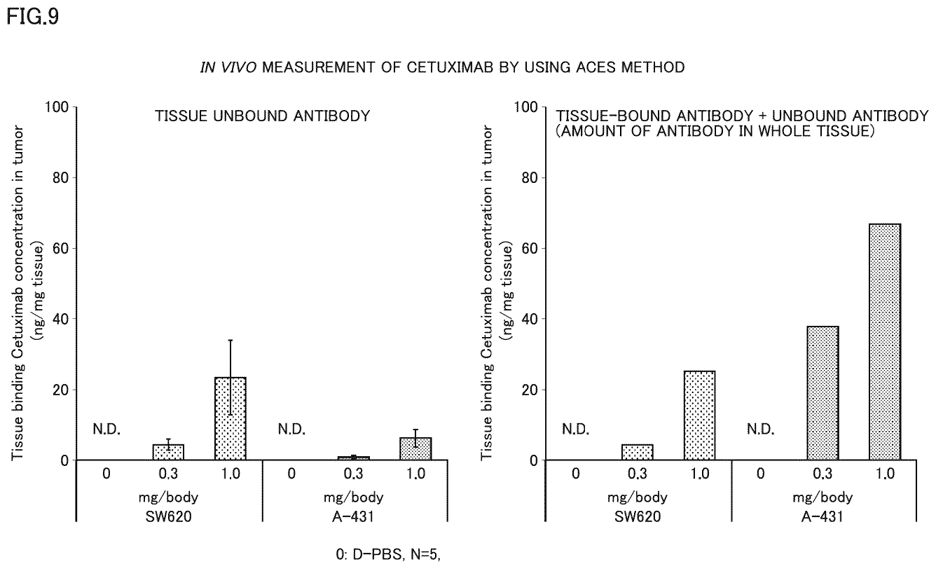

The sample corresponding to the “free antibody sample” in Experiment 1 was used to quantify cetuximab that was present in the model tumor but not bound to EGFR (FIG. 9, left side). More cetuximab was detected in model tumors from mice transplanted with the SW620 strain than in model tumors collected from mice transplanted with the A431 strain.

### Experiment 4: Extraction from Trastuzumab-Deruxtecan (T-DXd) Administered Cancer Tissue by ACES Method

### (Preparation Step, Extraction Step)

First, the following cancer tissue and preparation solution were provided.

### Cancer Tissue:

Cancer tissue from experimental model mice administered with T-DXd

The compositions of (1) tissue washing solution, (2) inhibitor solution, and (4) extraction solution were the same as those used in “Experiment 1: Quantifying antibody present in tissue sample”.

### (3) Internal Standard IS Solution:

### Inhibitor Solution:

SILuMAb Trastuzumab (final concentration 0.25 μg/ml)
BSA (final concentration 0.1%)
Mouse IgG1 (clone 2545) (final concentration 50 μg/ml)

First, the cancer tissue was sliced using a slicer to prepare thin sections (5 μm thick). Thin sections (10 mg) were transferred to a low-absorption microtube and treated in the same manner as in “Experiment 1: Quantifying antibody present in tissue sample”. In this manner, a sample containing crushed cancer tissue (homogenized biological sample) was prepared (preparation step). The biological sample is a sample derived from cancer cells. Thereafter, the “bound antibody sample” (analyte, or the first analyte) and the “free antibody sample” (the second analyte) were obtained from the sample containing the crushed cancer tissue by treating in the same manner as in “Experiment 1: Quantifying antibody present in tissue sample”.

### (Quantification Step)

The following preparation solutions were provided.

The compositions of (1) standard diluent, (2) washing solution, (3) neutralizing solution, and (4) reaction solution were the same as those solutions used in “Experiment 1: Quantifying antibody present in tissue sample”.

### (5) Initial Mobile Phase:

MilliQ water (0.1% acetic acid)+acetonitrile (volume ratio 99:1)

Standard curve samples and QC samples with the following compositions were provided.

The standard curve samples: all samples were prepared using a standard diluent.
Blank sample: 0 ng/ml Trastuzumab
Standard curve 1:250 ng/ml Trastuzumab
Standard curve 2:500 ng/ml Trastuzumab
Standard curve 3:1.00 μg/ml Trastuzumab
Standard curve 4:2.00 μg/ml Trastuzumab
Standard curve 5:4.00 g/ml Trastuzumab
Standard curve 6:8.0 μg/ml Trastuzumab
Standard curve 7:16.0 μg/ml Trastuzumab
Standard curve 8:32.0 μg/ml Trastuzumab
The QC samples: all samples were prepared using a standard diluent.
LLOQ: 250 ng/ml Trastuzumab
LQC: 750 ng/ml Trastuzumab
MQC: 2.50 μg/ml Trastuzumab
HQC: 12.0 g/ml Trastuzumab
HLOQ: 32.0 g/ml Trastuzumab

Here, 20 μL each of the corresponding standard curve sample or QC sample was added to 160 μL of matrix solution (a solution in which foreign components derived from the tissue for measurement were extracted in the same manner as for the sample for measurement while using an inhibitor solution and an extraction solution). An additional 20 μL of internal standard IS solution was added. These samples correspond to the “standard curve sample (with IS)” and “QC sample (with IS)” described below. In addition, in the standard curve sample preparation, samples further containing 20 μL of matrix solution instead of 20 μL of internal standard IS solution were also prepared. These samples correspond to the “standard curve sample (without IS)” described below.

The Trastuzumab (antigen-binding molecule) in each analyte was quantitatively analyzed by generating a peptide(s) specific for Trastuzumab (peptide(s) derived from the variable region) by the nSMOL method and detecting the peptide(s) with a mass spectrometer. Specifically, the procedure is as follows.

(Generating Peptides by nSMOL Method)

Bound antibody samples, standard curve samples (with or without IS), and QC samples (with IS) were each dispensed in 200 μl portions into low-absorption microtubes. Thereafter, these samples were processed in the same manner as in “Experiment 1: Quantifying antibody present in tissue sample”. In this manner, a peptide(s) specific for Trastuzumab (peptide(s) derived from the variable region) was generated.

### (Detecting Peptide(s) by Mass Spectrometry)

The peptide specific for Trastuzumab (SEQ ID NO: 2: IYPTNGYTR (one letter code)) was quantified by LCMS analysis under the conditions shown below.

### List of Solutions Prepared:

Mobile phase A: MilliQ water (0.1% acetic acid) (LCMS grade)
Mobile phase B: acetonitrile (LCMS grade)

### LCMS Analysis Conditions:

The analysis was performed in the same conditions as in “Experiment 1: Quantifying antibody present in tissue sample”.

### HPLC Conditions:

HPLC: Shimadzu Nexera X2
Flow rate: 0.4 ml/min
0-1.5 min: 1% B
1.5-5.5 min: 1-42% B gradient
5.5-6.5 min: 95% B
6.5-8.5 min: 1% B
LCMS analysis time: 2.5-6.5 min

### LCMS MRM Analysis Transition Conditions:

LCMS: Shimadzu LCMS-8060
Trastuzumab, IYPTNGYTR
542.90 (++)>404.80 (y7++) for quantification
542.90 (+++)>808.50 (y7+) confirmation ion
13C6, 15N4-Trastuzumab, 13C6, 15N4-IYPTNGYTR
547.90 (++)>409.70 (y7++) for quantification (Ab_IS)
547.90 (++)>818.50 (y7+) confirmation ion

### (Results)

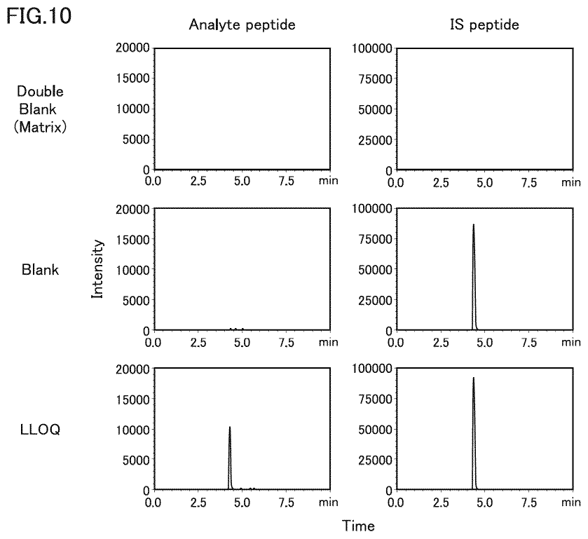

FIG. 10 shows the results of LCMS analysis of the standard curve sample (blank) (with IS (second graph from the top) or without IS (first graph from the top)) and the QC sample (LLOQ) (with IS (third graph from the top)) in the Example. The results of FIG. 10 demonstrated that Trastuzumab was detectable even at a concentration of 250 ng/ml under the analysis condition of the Example, as in the cetuximab Example.

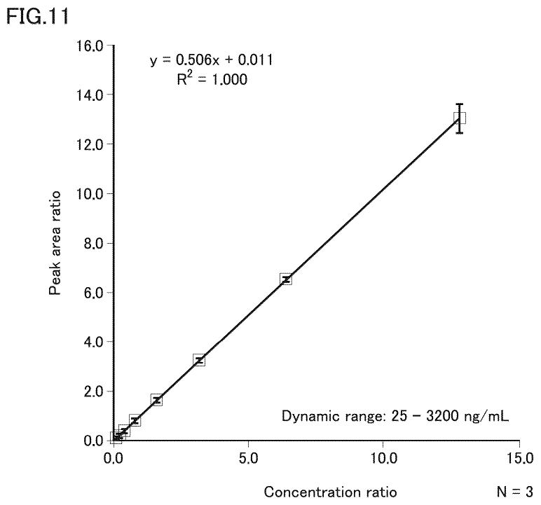

FIG. 11 shows a graph of the standard curve obtained from the results of analyzing the standard curve samples (blank, standard curves 1-8) (with IS) in the Example. In FIG. 11, the horizontal axis shows the concentration ratio of Trastuzumab in each standard curve sample (based on the concentration of IS), and the vertical axis shows the ratio of the peak area of analyte to the peak area of IS in the mass spectrometry. The results in FIG. 11 suggested that it was possible to quantify Trastuzumab under the analytical conditions of the Example.

### Experiment 5: Quantifying Antibody Portion (Trastuzumab) of ADC (T-DXd) Bound to Tissue in Model Tumor

Nude mice (BALB/c, female, 5-8 weeks old) were subcutaneously transplanted with SW620 or OE-19 strain. Here, SW620 strain is a cell line that does not express HER2 (antigen of trastuzumab), and OE-19 is a cell line that expresses HER2. At that time, the number of transplanted cells was 1×10⁷ cells per mouse. The mice were reared under normal conditions for 14 to 21 days until the transplanted cells engrafted. Then, T-DXd (0 or 10 mg/Kg) was injected intravenously into the mice. Twenty-four hours after intravenous injection, the transplanted cells (model tumors) were collected from mice as in “Experiment 3: Quantifying antibody bound to tissue in model tumor”.

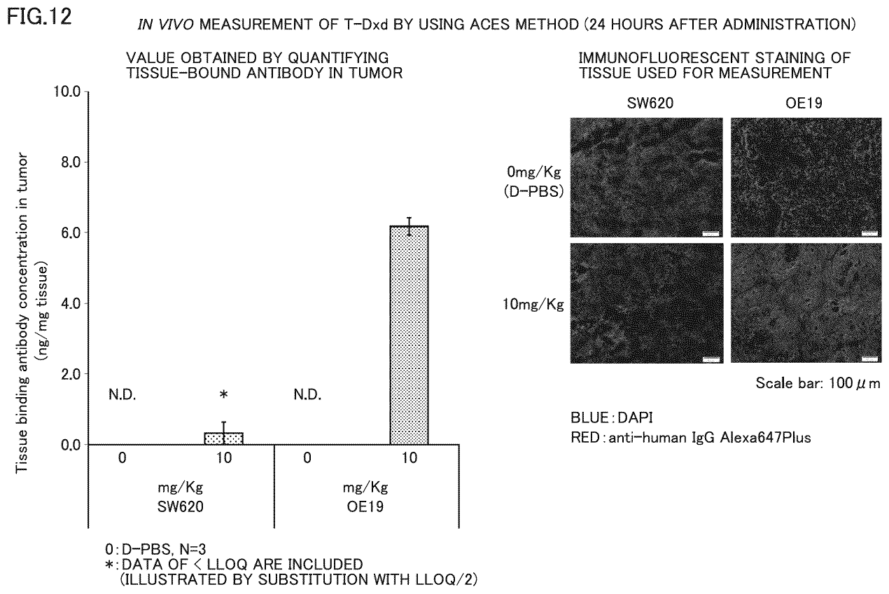

In addition, the collected model tumors were each tissue-stained with DAPI and anti-human IgG antibody (FIG. 12, right side). In model tumors from mice transplanted with the OE-19 strain, the antibody portion of T-DXd was stained in T-DXd-administered tissues.

The collected model tumors were treated by the same method as in Experiment 1, and the antibody portion of T-DXd bound to the model tumors was quantified. The samples used for quantification were those corresponding to the “bound antibody sample” in Experiment 1. FIG. 12 (left side) shows the results. In model tumors collected from mice transplanted with the OE-19 strain, Trastuzumab was detected only in T-DXd-administered tissues. On the other hand, Trastuzumab was hardly detectable in model tumors collected from mice implanted with the SW620 strain.

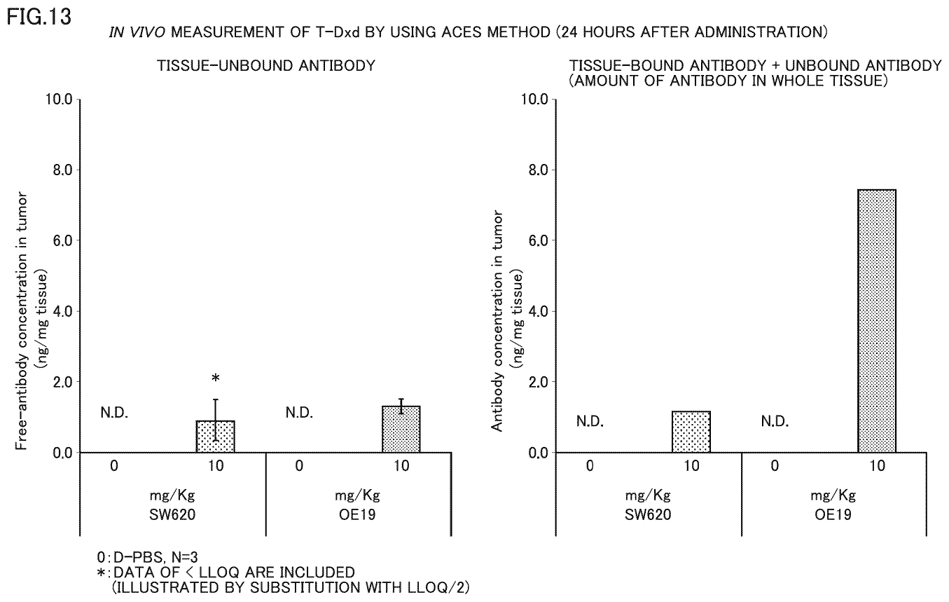

The sample corresponding to the “free antibody sample” in Experiment 4 was used to quantify the antibody portion of T-DXd that was present in the model tumor but not bound to HER2 (FIG. 13, left side). In model tumors collected from mice transplanted with the SW620 strain, the antibody portion of T-DXd was detected, as comparable to the model tumors obtained by transplanting the OE-19 strain (provided that the lower limit of quantification was calculated by substitution with half of the LLOQ value (12.5 ng/ml)).

### Experiment 6: Extraction from Trastuzumab-Administered Cancer Tissue by ACES Method

### (Preparation Step, Extraction Step)

First, the following cancer tissue and preparation solution were provided.

### Cancer Tissue:

Cancer tissue from experimental model mice administered with

### Trastuzumab

The compositions of (1) tissue washing solution, (2) inhibitor solution, and (4) extraction solution were the same as those used in “Experiment 1: Quantifying antibody present in tissue sample”.

### (3) Internal Standard IS Solution:

### Inhibitor Solution:

SILuMAb Trastuzumab (final concentration 0.25 μg/ml)
BSA (final concentration 0.1%)
Mouse IgG1 (clone 2545) (final concentration 50 μg/ml)

First, the cancer tissue was sliced using a slicer to prepare thin sections (5 μm thick). Thin sections (10 mg) were transferred to a low-absorption microtube and treated in the same manner as in “Experiment 1: Quantifying antibody present in tissue sample”. In this manner, a sample containing crushed cancer tissue (homogenized biological sample) (preparation step) was prepared. The biological sample is a sample derived from cancer cells. Thereafter, the “bound antibody sample” (analyte, or the first analyte) and the “free antibody sample” (the second analyte) were obtained from the sample containing the crushed cancer tissue by treating in the same manner as in “Experiment 1: Quantifying antibody present in tissue sample”.

### (Quantification Step)

The following preparation solutions were provided.

The compositions of (1) standard diluent, (2) washing solution, (3) neutralizing solution, and (4) reaction solution were the same as those solutions used in “Experiment 1: Quantifying antibody present in tissue sample”.

### (5) Initial Mobile Phase:

MilliQ water (0.1% acetic acid)+acetonitrile (volume ratio 99:1)

Standard curve samples and QC samples with the following compositions were provided.

The standard curve samples: all samples were prepared using a standard diluent.
Blank sample: 0 ng/ml Trastuzumab
Standard curve 1:250 ng/ml Trastuzumab
Standard curve 2:500 ng/ml Trastuzumab
Standard curve 3:1.00 μg/ml Trastuzumab
Standard curve 4:2.00 μg/ml Trastuzumab
Standard curve 5:4.00 μg/ml Trastuzumab
Standard curve 6:8.0 g/ml Trastuzumab
Standard curve 7:16.0 μg/ml Trastuzumab
Standard curve 8:32.0 μg/ml Trastuzumab
The QC samples: all samples were prepared using a standard diluent.
LLOQ: 250 ng/ml Trastuzumab
LQC: 750 ng/ml Trastuzumab
MQC: 2.50 μg/ml Trastuzumab
HQC: 12.0 μg/ml Trastuzumab
HLOQ: 32.0 μg/ml Trastuzumab

Here, 20 μL each of the corresponding standard curve sample or QC sample was added to 160 μL of matrix solution (a solution in which foreign components derived from the tissue for measurement were extracted in the same manner as for the sample for measurement while using an inhibitor solution and an extraction solution). An additional 20 μL of internal standard IS solution was added. These samples correspond to the “standard curve sample (with IS)” and “QC sample (with IS)” described below. In addition, in the standard curve sample preparation, samples further containing 20 μL of matrix solution instead of 20 μL of internal standard IS solution were also prepared. These samples correspond to the “standard curve sample (without IS)” and “QC sample (without IS)” described below.

The Trastuzumab (antigen-binding molecule) in each analyte was quantitatively analyzed by generating a peptide(s) specific for Trastuzumab (peptide(s) derived from the variable region) by the nSMOL method and detecting the peptide(s) with a mass spectrometer. Specifically, the procedure is as follows.

(Generating Peptides by nSMOL Method)

Bound antibody samples, standard curve samples (with or without IS), and QC samples (with or without IS) were each dispensed in 200 μl portions into low-absorption microtubes. Thereafter, these samples were processed in the same manner as in “Experiment 1: Quantifying antibody present in tissue sample”. In this manner, a peptide(s) specific for Trastuzumab (peptide(s) derived from the variable region) was generated.

### (Detecting Peptide(s) by Mass Spectrometry)

The peptide specific for Trastuzumab (SEQ ID NO: 2: IYPTNGYTR (one letter code)) was quantified by LCMS analysis under the conditions shown below.

### List of Solutions Prepared:

Mobile phase A: MilliQ water (0.1% acetic acid) (LCMS grade)
Mobile phase B: acetonitrile (LCMS grade)

### LCMS Analysis Conditions:

The analysis was performed in the same conditions as in “Experiment 1: Quantifying antibody present in tissue sample”.

### HPLC Conditions:

HPLC: Shimadzu Nexera X2
Flow rate: 0.4 ml/min
0-1.5 min: 1% B
1.5-5.5 min: 1-42% B gradient
5.5-6.5 min: 95% B
6.5-8.5 min: 1% B
LCMS analysis time: 2.5-6.5 min

### LCMS MRM Analysis Transition Conditions:

LCMS: Shimadzu LCMS-8060
Trastuzumab, IYPTNGYTR
542.90 (++)>404.80 (y7++) for quantification
542.90 (+++)>808.50 (y7+) confirmation ion
13C6, 15N4-Trastuzumab, 13C6, 15N4-IYPTNGYTR
547.90 (++)>409.70 (y7++) for quantification (Ab_IS)
547.90 (++)>818.50 (y7+) confirmation ion

### (Results)

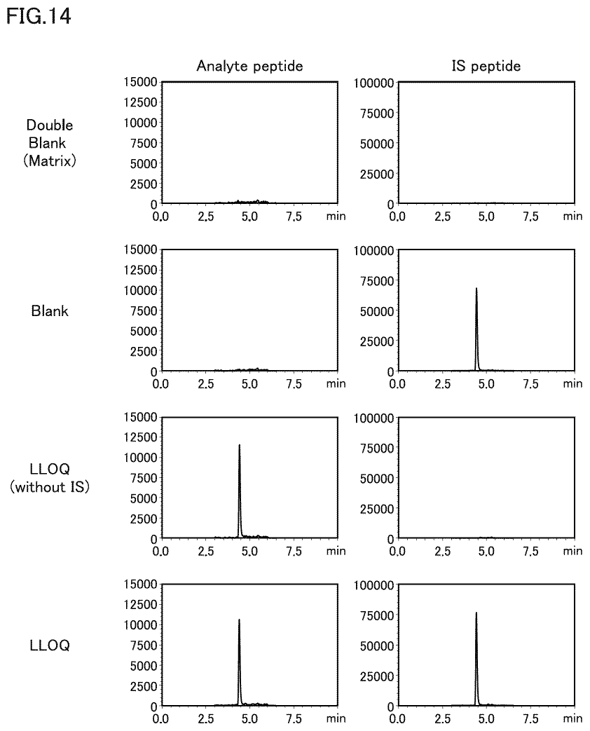

FIG. 14 shows the results of LCMS analysis of the standard curve sample (blank) (with IS (second graph from the top) or without IS (first graph from the top)) and the QC sample (LLOQ) (with IS (fourth graph from the top) or without IS (third graph from the top)) in the Example. The results of FIG. 14 demonstrated that Trastuzumab was detectable even at a concentration of 250 ng/ml under the analysis condition of the Example, as in the cetuximab Example.

A graph (not shown) was drawn for the standard curve obtained from the results of analyzing the standard curve samples (blank, standard curves 1-8) (with IS) in the Example. The results of this graph have suggested that Trastuzumab can be quantified under the analytical conditions of the Example.

### Experiment 7: Quantifying Trastuzumab Bound to Tissue in Model Tumor

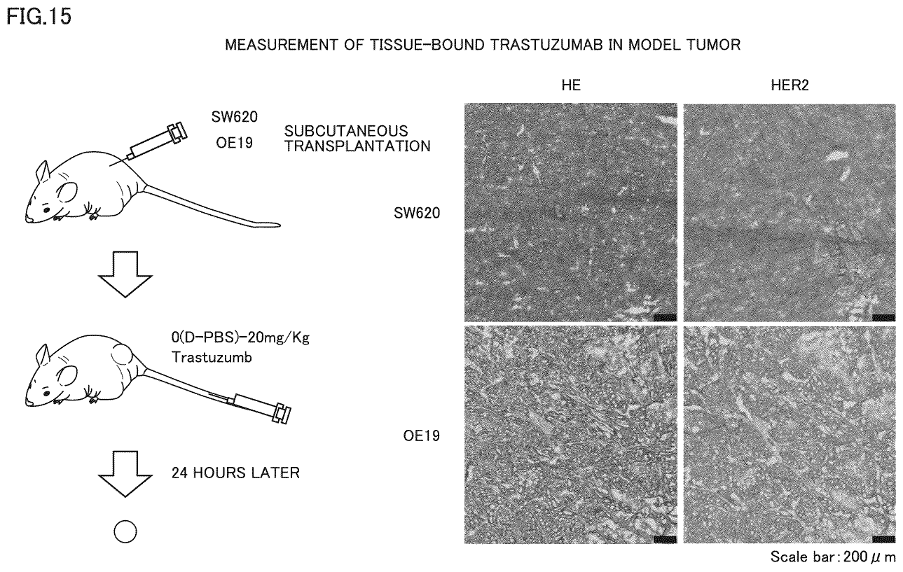

Nude mice (BALB/c, female, 5-8 weeks old) were subcutaneously transplanted with SW620 or OE-19 strain. Here, SW620 strain is a cell line that does not express HER2 (antigen of trastuzumab), and OE-19 is a cell line that expresses HER2. At that time, the number of transplanted cells was 1×10⁷ cells per mouse. The mice were reared under normal conditions for 14 to 21 days until the transplanted cells engrafted. Then, Trastuzumab (0, 10, or 20 mg/Kg) was injected intravenously into the mice. Twenty-four hours after intravenous injection, the transplanted cells (model tumors) were collected from mice as in “Experiment 3: Quantifying antibody bound to tissue in model tumor” (FIG. 15, left side).

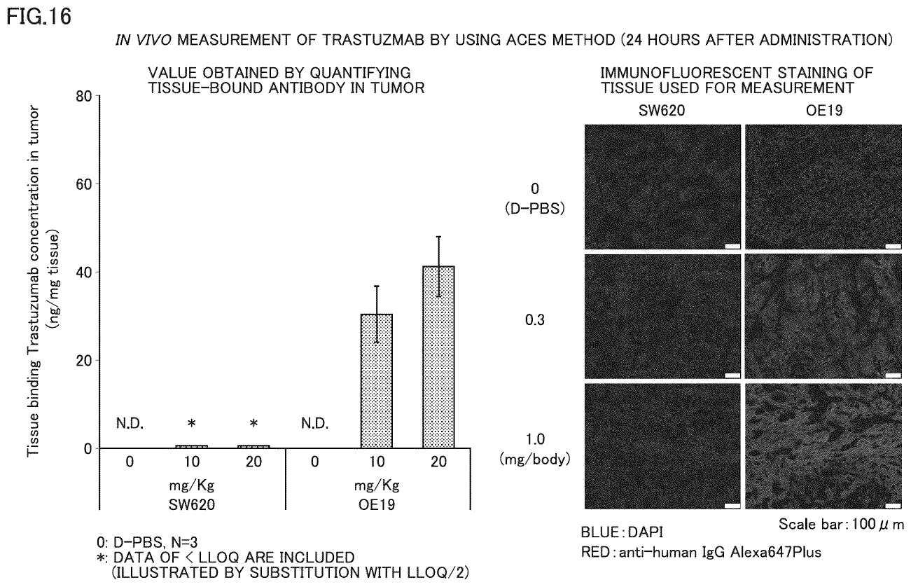

In addition, the collected model tumors were each tissue-stained with DAPI and anti-human IgG antibody (FIG. 16, right side). In model tumors collected from mice transplanted with the OE-19 strain, Trastuzumab was stained in Trastuzumab-administered tissues.

The collected model tumors were treated by the same method as in Experiment 1, and the antibody portion of Trastuzumab bound to the model tumors was quantified. The samples used for quantification were those corresponding to the “bound antibody sample” in Experiment 1. FIG. 16 (left side) shows the results. In model tumors collected from mice implanted with the OE-19 strain, Trastuzumab was detected only in Trastuzumab-administered tissue. On the other hand, Trastuzumab was hardly detectable in model tumors collected from mice implanted with the SW620 strain.

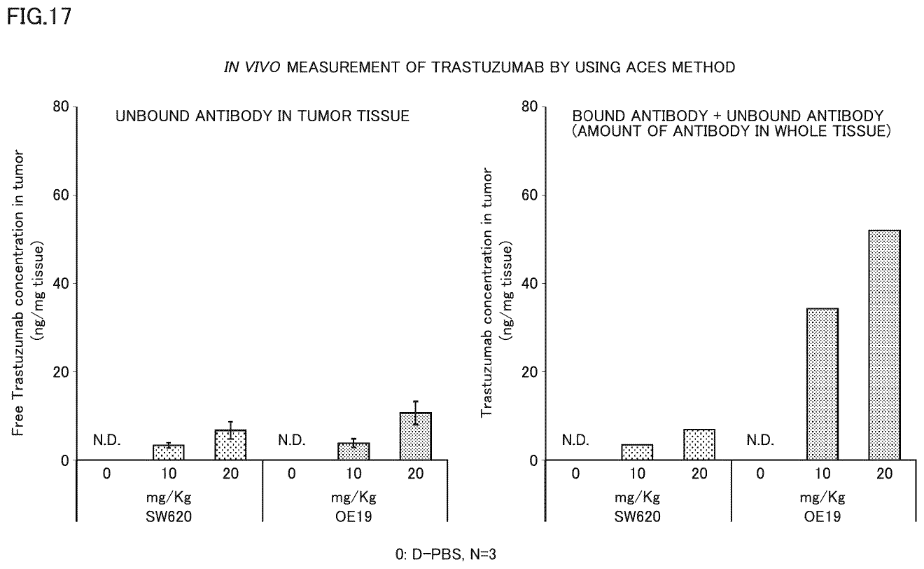

The sample corresponding to the “free antibody sample” in Experiment 6 was used to quantify Trastuzumab that was present in the model tumor but not bound to HER2 (FIG. 17, left side). In model tumors collected from mice transplanted with the SW620 strain, the Trastuzumab was detected, as comparable to the model tumors obtained by transplanting the OE-19 strain (provided that the lower limit of quantification was calculated by substitution with half of the LLOQ value (12.5 ng/ml)).

### [Items]

It is understood by those skilled in the art that the-described plurality of exemplary Embodiments and Examples are specific examples of the following items.

### (Item 1)

A method of quantifying an antigen-binding molecule of one aspect is a method of quantifying an antigen-binding molecule bound to cells, comprising: a preparation step of preparing a biological sample derived from the cells, wherein the biological sample is homogenized, the cells express a target antigen bound specifically to the antigen-binding molecule, and the cells have been exposed to the antigen-binding molecule; an extraction step of adding an extraction solution to the biological sample to obtain an analyte, wherein the extraction solution comprises an organic acid and a nonionic surfactant, and the extraction solution has a pH of 1 or more and 3 or less; and a quantification step of analyzing the analyte to quantify the antigen-binding molecule. The quantifying method according to item 1 enables accurate quantification of an antigen-binding molecule bound to cells expressing its target antigen.

### (Item 2)

In the method according to item 1, the organic acid comprises at least one selected from the group consisting of arginine, citrulline, and glycine. The method according to item 2 enables extraction of the antigen-binding molecule in a stable condition.

### (Item 3)

In the method according to item 1 or 2, the nonionic surfactant comprises an alkyl glycoside. The method according to item 3 enables extraction of the antigen-binding molecule in a stable condition.

### (Item 4)

In the quantifying method according to any one items 1 to 3, the target antigen comprises at least one selected from the group consisting of EGFR, HER2, CD30, PD-L1, RANKL, tumor necrosis factor receptors, insulin receptor, and vascular endothelial growth factor receptors. The method according to item 4 enables accurate quantification of the antigen-binding molecule bound to a given target antigen.

### (Item 5)

In the method according to any one of items 1 to 4, the antigen-binding molecule comprises an antibody or a peptide aptamer. The method according to item 5 enables accurate quantification of the antibody or the peptide aptamer.

### (Item 6)

In the method according to the item 5, the antigen-binding molecule comprises at least one selected from the group consisting of cetuximab, trastuzumab, brentuximab, denosumab, infliximab, adalimumab, etanercept, aflibercept, ramucirumab, atezolizumab, avelumab, and insulin analogs. The method according to item 6 enables accurate quantification of a given antibody.

### (Item 7)

In the method according to any one of items 1 to 6, the cells are cells derived from cancer tissue, or cells derived from diseased tissue. The method according to item 7 enables accurate quantification of the amount of antigen-binding molecule accumulated in cancer tissue or diseased tissue.

### (Item 8)

The quantifying method according to any one of items 1 to 7, wherein the antigen-binding molecule is an antibody; and the quantification step comprises generating a peptide derived from a variable region of the antibody by nSMOL method and analyzing the peptide by using an LC-MS analyzer. The method according to item 8 further enables accurate quantification of the antibody.

The Embodiments and Examples of the present invention have been described above. However, it is also originally contemplated to combine the above-mentioned features of Embodiments and Examples as appropriate.

The Embodiments and Examples disclosed herein are illustrative in all respects and should not be considered to be limited. The scope of the present invention is defined by the Claims, but not by the Embodiments and Examples described above, and it is intended that all the modifications within the meanings and scope of the Claims and their equivalents are encompassed thereby.

What is claimed is:

1. A method of quantifying an antigen-binding molecule bound to cells, comprising:
a preparation step of preparing a biological sample derived from the cells, wherein
the biological sample is homogenized,
the cells express a target antigen bound specifically to the antigen-binding molecule, and
the cells have been exposed to the antigen-binding molecule;
an extraction step of adding an extraction solution to the biological sample to obtain an analyte, wherein
the extraction solution comprises an organic acid and a nonionic surfactant, and
the extraction solution has a pH of 1 or more and 3 or less; and
a quantification step of analyzing the analyte to quantify the antigen-binding molecule.

2. The method according to claim 1, wherein the organic acid comprises at least one selected from the group consisting of arginine, citrulline, and glycine.

3. The method according to claim 1, wherein the nonionic surfactant comprises an alkyl glycoside.

4. The method according to claim 1, wherein the target antigen comprises at least one selected from the group consisting of EGFR, HER2, CD30, PD-L1, RANKL, tumor necrosis factor receptors, insulin receptor, and vascular endothelial growth factor receptors.

5. The method according to claim 1, wherein the antigen-binding molecule comprises an antibody or a peptide aptamer.

6. The method according to claim 5, wherein the antigen-binding molecule comprises at least one selected from the group consisting of cetuximab, trastuzumab, brentuximab, denosumab, infliximab, adalimumab, etanercept, aflibercept, ramucirumab, atezolizumab, avelumab, and insulin analogs.

7. The method according to claim 1, wherein the cells are cells derived from cancer tissue, or cells derived from diseased tissue.

8. The method according to claim 1, wherein the antigen-binding molecule is an antibody; and
the quantification step comprises generating a peptide derived from a variable region of the antibody by nSMOL method and analyzing the peptide by using an LC-MS analyzer.


SEQUENCE LISTING (2 sequences)

SEQ ID NO: 1 - protein, 15 aa, synthetic construct
  source 1..15: note = Surrogate peptide of Cetuximab
ASQSIGTNIHWYQQR

SEQ ID NO: 2 - protein, 9 aa, synthetic construct
  source 1..9: note = Surrogate peptide of Trastuzumab
IYPTNGYTR